(19) United States
(12) Patent Application Publication
(10) Pub. No.: US 2023/0346763 A1
(43) Pub. Date: Nov. 2, 2023

(54) METHOD FOR PREPARING SOLID FORMULATION OF PIMAVANSERIN

(71) Applicant: NANJING KAIWANG PHARMACEUTICAL CO., LTD., Nanjing (CN)

(72) Inventors: Shaoping WANG, Nanjing (CN); Weining YANG, Los Angeles, CA (US)

(21) Appl. No.: 18/027,840

(22) PCT Filed: Sep. 19, 2021
(86) PCT No.: PCT/CN2021/119473

§ 371 Date: Mar. 22, 2023

(30) Foreign Application Priority Data
Sep. 23, 2020 (CN) 202011014255.5

(51) Int. Cl. A61K 31/4468; A61K 9/48; A61K 9/16

(52) U.S. Cl. CPC A61K 31/4468; A61K 9/4833; A61K 9/4866; A61K 9/4858; A61K 9/16; A61K 9/1688

(57) ABSTRACT

A method for a pimavanserin solid preparation. The method improves the fluidity of pimavanserin powder during preparation, can improve the stability of the content of an active ingredient in the solid preparation, and has the characteristics of rapid dissolution and release and good compression moldability.

### TECHNICAL FIELD

The present invention relates to a method for a pimavanserin solid preparation. The method improves the fluidity of pimavanserin powder during preparation, can improve the stability of the content of the active ingredient in the solid preparation, and has the characteristics of rapid dissolution and release and good compression moldability.

### BACKGROUND ART

Pimavanserin, the structure of which is as shown below, is a selective 5-hydroxytryptamine 2A/2C receptor inverse agonist and is described in International Patent Application PCT/US2004/001234. A tartrate thereof, pimavanserin tartrate, was approved for sale on the market by FDA on Apr. 29, 2016 (Nuplazid®) against mental diseases (hallucinations and delusions) complicated by Parkinson's disease.

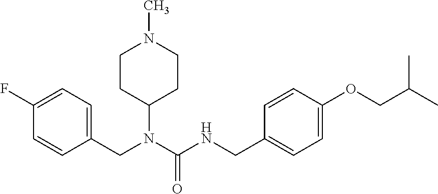 [SMILES: CC(C)COc1ccc(CNC(=O)N(Cc2ccc(F)cc2)C2CCN(C)CC2)cc1]

Pimavanserin is usually prepared into solid preparations such as tablets and capsules. For example, the drug pimavanserin tartrate on the market in the United States exists in the form of tablets and capsules. Such solid preparations are usually prepared by mixing the active ingredient with appropriate excipients to make granules, and then pressing a certain amount of granules (to make tablets) or filling them in capsules (to make capsules). During the industrial production of solid preparations, in order to ensure that the content of the active ingredient in the prepared granules and the tablets or capsules thus prepared is uniform and stable, it is necessary to improve the fluidity of powder during the mixing of the active ingredient with the excipients.

It is well known to those skilled in the art that particle size greatly affects the fluidity of powder during the preparation of solid preparations. The larger the particle size of the powder, the smaller the angle of repose generally and also the higher the fluidity. Generally, if the particle size is less than 200 μm, the particles tend to aggregate and show adhesiveness (see Pharmaceutics, edited by ZHANG, Qiang and W U, Fenglan, 1st edition, 2005, Peking University Medical Press). However, due to the special inherent properties of pimavanserin tartrate molecules, the powder fluidity of the active ingredient is very poor, and it is difficult to prepare granules with uniform content even by technical means commonly used in the art such as increasing the particle size of the powder and adding a lubricant, so that the content stability of the prepared solid preparation is relatively low, and it is difficult to ensure the accuracy of dosage in clinical applications.

In addition to the need to improve the fluidity of the powder, in order to ensure the absorption of the active ingredient, the active ingredient in the solid preparation should also have a certain degree of dissolution; in addition, in the case of the preparation of tablets, the pressed tablets should also be easy to compress and form and not easy to damage, thus facilitating production and transportation links and maintaining the stability of drug quality. Most of the existing pimavanserin tartrate solid preparations cannot yet reach a satisfactory level in terms of dissolution and compression moldability.

To sum up, there is an urgent need in the art for a new method for preparing a pimavanserin solid preparation (especially pimavanserin tartrate solid preparation), in order to improve the powder fluidity of pimavanserin during preparation as well as the dissolution and compression moldability.

### SUMMARY OF THE INVENTION

By a great deal of in-depth research on solid preparations of pimavanserin, especially pimavanserin tartrate, the inventors of the present invention have unexpectedly found that contrary to the operation of increasing the particle size as commonly used in the prior art, the particle size of a powder of the bulk drug pimavanserin is controlled to be 178 μm (equivalent to 80 mesh) or less by further crushing the powder, which, on the contrary, could significantly improve the fluidity of the powder during granulation, improve the content stability of the preparation, and can improve the compression moldability of the preparation and the dissolution of the active ingredient, thus completing the invention.

Therefore, the present invention relates to a method for preparing a pimavanserin solid preparation, the method comprising the following steps:
(1) crushing the bulk drug pimavanserin into a particle size of 178 μm or less;
(2) mixing the crushed bulk drug pimavanserin with a pharmaceutically acceptable excipient;
(3) dry granulating the mixed powder; and
(4) pressing the prepared granules into tablets or filling the prepared granules into capsules.

In a preferred technical solution, the pimavanserin is pimavanserin tartrate. In a preferred technical solution, the pimavanserin is pimavanserin in a crystalline form. In a more preferred technical solution, the crystalline form is pimavanserin crystal form C as described in International Patent Application PCT/US2004/001234.

In a preferred technical solution, the particle size is expressed as D90, preferably D95, more preferably D98.

In a preferred technical solution, in step (1), the bulk drug pimavanserin is crushed into a particle size of 170 μm or less, 160 μm or less, or 150 μm or less.

In a preferred technical solution, the excipient includes a filler and a lubricant. In a more preferred technical solution, the filler is microcrystalline cellulose. In a more preferred technical solution, the lubricant is magnesium stearate.

In a more preferred technical solution, the mass ratio of the bulk drug pimavanserin to the filler to the lubricant is (20-60):(40-80):(0.1-5), preferably (30-50):(50-70):(0.2-2), more preferably about 40:59:(0.5-1).

In a preferred technical solution, the mixing is carried out using a mixer. In a more preferred technical solution, the mixer is a three-dimensional mixer, a V-shaped mixer, a trough mixer, a square cone hopper mixer, and more preferably a hopper mixer.

In a more preferred technical solution, the rotational speed of the mixer is about 1-100 rpm, preferably 2-50 rpm, more preferably about 5-20 rpm (for example, about 10 rpm); and the mixing time is about 1-60 minutes, preferably 2-30 minutes, and more preferably about 5-20 minutes (for example, about 10 minutes).

In a preferred technical solution, the dry granulation is carried out using a dry granulator, and the granulation pressure is about 1-100 bar, preferably 20-80 bar, more preferably 40-60 bar, and particularly preferably, the granulation pressure is about 50 bar.

In a preferred technical solution, after step (3), the following step (3′) is carried out:

(3′) mixing the prepared granules with a second portion of pharmaceutically acceptable excipient, wherein the second portion of pharmaceutically acceptable excipient comprises a second portion of lubricant (such as magnesium stearate), wherein the mass ratio of the bulk drug pimavanserin to the second portion of lubricant is (20-60):(0.1-5), preferably (30-50):(0.2-2), more preferably about 40:59:0.5.

In a preferred technical solution, the particle size D90 of the prepared granules is 100-1000 μm, preferably 200-800 μm, more preferably 300-600 μm.

In a preferred technical solution, the solid preparation is in the form of tablets or capsules, preferably capsules, and is characterized in that the capsule shell is preferably selected from gelatin capsules, potato capsules, plant (hydroxypropyl methylcellulose) capsules, sodium alginate capsules, more preferably plant (hydroxypropyl methylcellulose) capsules.

The method of the present invention improves the fluidity of pimavanserin powder during preparation, can improve the stability of the content of an active ingredient in the solid preparation, and has the characteristics of rapid dissolution and release and good compression moldability. Therefore, the preparation and method of the present invention are not only suitable for operation and control in large-scale industrial production but can also significantly improve the stability of the dosage and facilitate the active ingredient to exert efficacy, thus benefiting patients. In particular, the method of the present invention has no special requirements on the density and particle size of auxiliary materials, and it is only necessary to use conventional microcrystalline cellulose and magnesium stearate as a filler and a lubricant respectively, thus greatly reducing the cost of the preparation. In addition, the method of the present invention has higher process durability and reproducibility, greatly reduces the requirements for production equipment and process control, has better equipment applicability, and also greatly reduces the risk of process parameter control.

### BRIEF DESCRIPTION OF THE DRAWINGS

FIG. 1: a dissolution curve of the pimavanserin tartrate capsules prepared according to the method of the present invention in 0.1 mol/L hydrochloric acid solution.

FIG. 2: a dissolution curve of the pimavanserin tartrate capsules prepared according to the method of the present invention in pH 4.5 acetic acid-sodium acetate buffer solution.

FIG. 3: a dissolution curve of the pimavanserin tartrate capsules prepared according to the method of the present invention in water.

FIG. 4: a dissolution curve of the pimavanserin tartrate capsules prepared according to the method of the present invention in pH 6.8 phosphate buffer solution.

FIG. 5: dissolution curve of the commercial drug Nuplazid® in 0.1 mol/L hydrochloric acid solution.

### DETAILED DESCRIPTION OF EMBODIMENTS

The term “about” used in the context of the description represents a range of 10% fluctuation from the corresponding value. For example, if the concentration of a certain ingredient is about 5 mM, the concentration thereof is indicated to be 4.5-5.5 mM; and if the concentration range of a certain ingredient is about 5-10 mM, the concentration range thereof is indicated to be 4.5-11 mM.

Unless otherwise specified, the term “active ingredient” in the context of the description refers to pimavanserin, especially pimavanserin tartrate.

The term “particle size” used in the context of the description generally refers to the particle size with a particle cumulative distribution of 90%, i.e., “D90”. For example, the expression “particle size is 178 μm or less” in the context of the description generally means that “D90 is 178 μm or less”. Preferably, the term “particle size” refers to the particle size with a particle cumulative distribution of 95%, i.e., “D95”; more preferably, the term “particle size” refers to the particle size with a particle cumulative distribution of 98%, i.e., “D98”.

The term “bulk drug” used in the context of the description refers to the raw material of pimavanserin, which has not been prepared into a preparation, the form of which includes but is not limited to amorphous powder and crystal powder, and the purity of which meets the requirements of pharmacopoeia or other normative documents or meets the general requirements in the pharmaceutical field for bulk drugs. The source thereof is commercially available or obtained by chemical synthesis by researchers themselves.

The terms “or more” and “or less” used in the context of the description are inclusive. For example, “particle size is 178 μm or less” and “particle size is less than or equal to 178 μm” have the same meaning.

One specific embodiment of the present invention relates to a method for preparing a pimavanserin solid preparation, the method comprising the following steps:
(1) crushing a tartrate of the bulk drug pimavanserin into a D90 (preferably D95, more preferably D98) of 178 μm or less (for example, by passing the tartrate of the bulk drug pimavanserin through an 80-mesh sieve);
(2) mixing the crushed tartrate of the bulk drug pimavanserin with a pharmaceutically acceptable excipient using a hopper mixer, wherein the pharmaceutically acceptable excipient includes microcrystalline cellulose and magnesium stearate; the mass ratio of the bulk drug pimavanserin to microcrystalline cellulose to magnesium stearate is (20-60):(40-80):(0.1-5), preferably (30-50):(50-70):(0.2-2), more preferably about 40:59:(0.5-1); the rotational speed of the mixer is about 1-100 rpm, preferably 2-50 rpm, more preferably about 5-20 rpm (for example, about 10 rpm); and the mixing time is about 1-60 minutes, preferably 2-30 minutes, and more preferably about 5-20 minutes (for example, about 10 minutes);
(3) dry granulating the mixed powder using a dry granulator, wherein the granulation pressure of the dry granulator is about 1-100 bar, preferably 20-80 bar, more preferably 40-60 bar, and particularly preferably about 50 bar;
(3′) optionally mixing the prepared granules with a second portion of magnesium stearate, wherein the mass ratio of the bulk drug pimavanserin to the second portion of magnesium stearate is (20-60):(0.1-5), preferably (30-50):(0.2-2), more preferably about 40:59:0.5; and
(4) pressing the prepared granules into tablets or filling the prepared granules into capsules.

The more specific embodiments of the present invention will be explained and described in an illustrative manner by the following examples; however, it should be recognized that the examples are not intended to limit the scope of the present invention.

### Example 1: Capsule Preparation Process 1

The tartrate of the bulk drug pimavanserin was prepared according to the method described in International Patent Application PCT/US2004/001234. The bulk drug was passed through an 80-mesh sieve to control the particle size (D90) thereof to be 178 μm or less. It was then weighed on a proportional basis according to the prescribed amounts shown in Table 1 below.

TABLE 1
Prescribed amounts for preparation process 1

| Prescription | Amount (mg/capsule) | Function |
|---|---|---|
| Pimavanserin tartrate | 40 | Active ingredient |
| Microcrystalline | 59 | Filler |
| cellulose | | |
| Magnesium stearate | 1 | Lubricant |

Pimavanserin tartrate, microcrystalline cellulose, and magnesium stearate, which satisfied the above ratio, were added to a hopper mixer; with the rotating speed being set to 10 rpm and the time to 10 minutes, the mixer was started for mixing. After the mixing was complete, the mixed powder was added to a dry granulator for dry granulation, wherein the granulation pressure of the dry granulator was set to 50 bar. After granulation, the granules were filled into size 3 plant capsules, and a total of 5000 capsules were prepared.

### Example 2: Capsule Preparation Process 2

The tartrate of the bulk drug pimavanserin was passed through an 80-mesh sieve to control the particle size (D90) thereof to be 178 μm or less. It was then weighed on a proportional basis according to the prescribed amounts shown in Table 2 below.

TABLE 2
Prescribed amounts for preparation process 2

| Prescription | Amount (mg/capsule) | Function |
|---|---|---|
| Pimavanserin tartrate | 40 | Active ingredient |
| Microcrystalline cellulose | 59 | Filler |
| Magnesium stearate | 0.5 | Lubricant |
| (added internally) | | |
| Magnesium stearate | 0.5 | Lubricant |
| (added externally) | | |

Pimavanserin tartrate, microcrystalline cellulose, and magnesium stearate (added internally), which satisfied the above ratio, were added to a hopper mixer; with the rotating speed set to 10 rpm and the time to 10 minutes, the mixer was started for mixing. After the mixing was complete, the mixed powder was added to a dry granulator for dry granulation, wherein the granulation pressure of the dry granulator was set to 50 bar. After granulation, the prepared granules and magnesium stearate (added externally) at 0.5 mg/capsule were added to the mixer; and with the rotation speed being set to 10 rpm and the time to 10 minutes. the mixer was started for mixing until uniform, and the mixed material was filled into size 3 plant capsules.

### Example 3: Evaluation of Fluidity and Particle Size Distribution of Granules

The angle of repose, bulk density, and tap density of the capsule granules of Example 2 were measured. The determination method was as follows, and the results were as shown in Table 3.

Angle of repose: A funnel was fixed at a certain height above a piece of graph paper, and a material was added from the funnel until the bottom of the formed accumulated cone just came into contact with the bottom of the funnel, the diameter of the cone was measured, and the angle of repose was calculated by taking the ratio of the height of the bottom of the funnel to the radius of the cone as the tangent value (measured in parallel three times).

Bulk density: About 100 g of the uncompacted material to be measured was added to a dry 250 ml measuring cylinder (readable to 2 mL), with the weighing accuracy being 0.1%, and the sample was carefully scraped flat without compaction; and the initial volume VO was read, i.e., to the nearest calibration value, and the packing density (g/ml) was obtained. The calculation formula was m/V0 (measured three times in parallel).

Tap density: The measuring cylinder was fixed on a bracket. It was tapped 10, 500, and 1250 times, and the corresponding volumes V10, V500 and V1250 were read to the nearest calibration values. When the difference between V500 and V1250 was less than or equal to 2 ml, V1250 was the tap volume VF. When the difference between V500 and V1250 exceeded 2 ml, it was tapped 1250 times until the difference between the measured values was less than or equal to 2 ml, and the read volume was the final tap volume VF. The calculation formula was m/VF (measured three times in parallel).

TABLE 3
Measurement results of the fluidity of capsule granules

| | Angle of repose | Bulk density | Tap density |
|---|---|---|---|
| Test No. | (°) | (g/cm³) | (g/cm³) |
| 1 | 30.2 | 0.50 | 0.58 |
| 2 | 31.5 | 0.49 | 0.58 |

In addition, in order to evaluate the particle size distribution of the prepared capsule granules, different sieves were used for sieving, and the results thereof were as shown in Table 4:

TABLE 4
Particle size distribution of capsule granules

| | Aperture (μm) | Proportion (wt %) |
|---|---|---|
| 20 mesh | 850 | 0.24 |
| 30 mesh | 600 | 56.5 |
| 45 mesh | 355 | 27.1 |
| 60 mesh | 250 | 11.3 |

From the above, it was indicated that the granules prepared in Examples 1 and 2 had better fluidity and also more rational particle size distribution, and were suitable for capsule filling. The stability of the filling dose could be ensured, and during the continuous processing of material transfer and filling in production, the stability of the content uniformity of the bulk drug could also be ensured, and the accuracy of therapeutic dose was finally ensured.

### Example 4: Evaluation of Content Uniformity

10 capsules obtained in Example 2 were randomly selected and numbered 1-10 respectively, and the relative value of the content of the active ingredient therein was determined, with 40 mg as 100%. As shown in Table 5, the distribution of the active ingredient had good uniformity, indicating that the content was stable.

TABLE 5

| Capsule No. | Content (%) | RSD (%) |
|---|---|---|
| 1 | 87.5 | 1.7% |
| 2 | 90.6 | |
| 3 | 99.5 | |
| 4 | 99.9 | |
| 5 | 101.2 | |
| 6 | 100 | |
| 7 | 99.2 | |
| 8 | 93.8 | |
| 9 | 89.1 | |
| 10 | 88.9 | |

### Example 5: Evaluation of Dissolution

The dissolution of the capsules of Example 2 was determined:

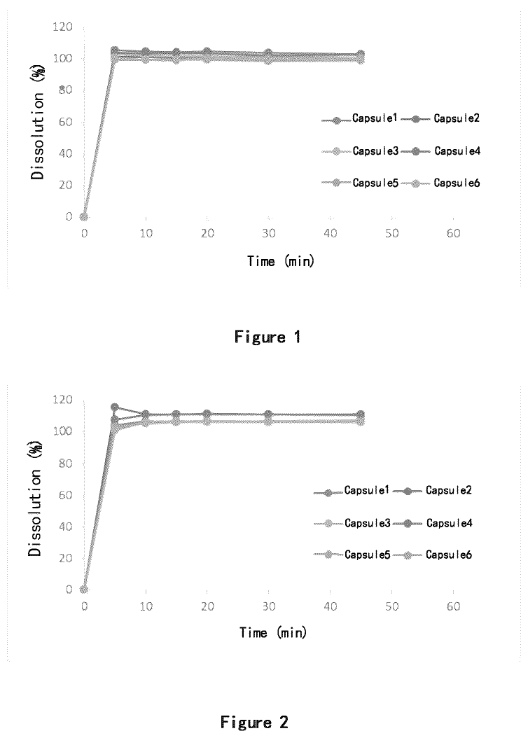

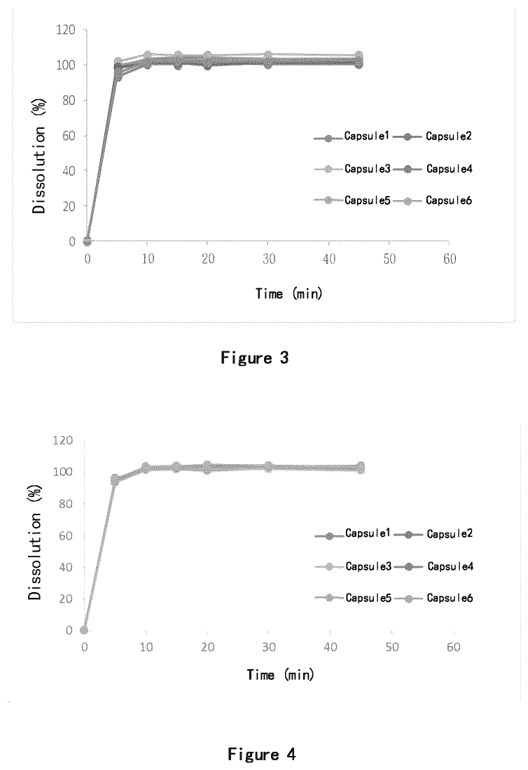

Experiments were carried out in 900 ml dissolution media at a speed of 100 rpm by a basket method. The dissolution media were respectively 0.1 mol/L hydrochloric acid solution, pH 4.5 acetic acid-sodium acetate buffer solution, water, and pH 6.8 phosphate buffer solution. Six experiments were conducted in parallel for each dissolution medium according to the method described in the FDA review (NDA210793 Product Quality Review). Dissolution was measured at 5, 10, 15, 20, 30 and 45 minutes after the start of the experiment, and the results were separately as shown in FIGS. 1-5 and Tables 6-10.

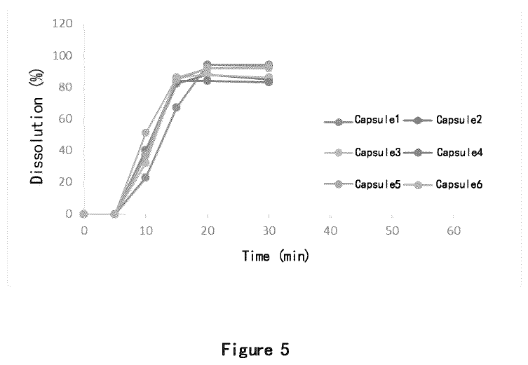

For comparison, FIG. 5 and Table 10 showed the dissolution test results of Nuplazid®, a commercially available pimavanserin tartrate capsule as mentioned in the above FDA review, and the dissolution medium used was 0.1 mol/L hydrochloric acid solution.

TABLE 6
Dissolution curve of pimavanserin tartrate capsules
in 0.1 mol/L hydrochloric acid solution

| | Dissolution (%) | | | | | |
|---|---|---|---|---|---|---|
| No. | 5 min | 10 min | 15 min | 20 min | 30 min | 45 min |
| 1 | 105.68 | 104.69 | 104.02 | 104.43 | 103.54 | 102.94 |
| 2 | 100.85 | 100.99 | 100.77 | 100.98 | 101.17 | 99.95 |
| 3 | 102.96 | 101.98 | 101.42 | 101.57 | 100.65 | 100.22 |
| 4 | 103.81 | 103.15 | 103.23 | 103.30 | 101.74 | 102.37 |
| 5 | 99.23 | 99.48 | 99.11 | 99.17 | 98.26 | 98.60 |
| 6 | 100.51 | 99.29 | 98.88 | 100.28 | 99.70 | 99.99 |
| Mean (%) | 102.7 | 101.59 | 101.24 | 101.62 | 100.84 | 100.68 |
| RSD (%) | 2.3 | 2.1 | 2.1 | 1.9 | 1.8 | 1.6 |

TABLE 7
Dissolution curve of pimavanserin tartrate capsules
in pH 4.5 acetic acid-sodium acetate buffer solution

| | Dissolution (%) | | | | | |
|---|---|---|---|---|---|---|
| No. | 5 min | 10 min | 15 min | 20 min | 30 min | 45 min |
| 1 | 103.76 | 106.21 | 106.12 | 106.36 | 106.27 | 106.91 |
| 2 | 115.42 | 110.78 | 110.87 | 110.80 | 110.82 | 110.93 |
| 3 | 101.95 | 105.59 | 106.13 | 106.10 | 105.96 | 105.92 |
| 4 | 107.06 | 110.29 | 110.73 | 111.04 | 110.95 | 110.49 |
| 5 | 102.62 | 105.10 | 105.86 | 106.21 | 105.94 | 106.00 |
| 6 | 100.74 | 105.89 | 106.50 | 106.26 | 106.17 | 106.28 |
| Mean (%) | 105.26 | 107.70 | 107.80 | 107.80 | 107.68 | 107.75 |
| RSD (%) | 5.2 | 2.4 | 2.2 | 2.2 | 2.3 | 2.2 |

TABLE 8
Dissolution curve of pimavanserin tartrate capsules in water

| | Dissolution (%) | | | | | |
|---|---|---|---|---|---|---|
| No. | 5 min | 10 min | 15 min | 20 min | 30 min | 45 min |
| 1 | 98.96 | 103.04 | 103.70 | 103.88 | 103.45 | 103.33 |
| 2 | 93.29 | 100.57 | 100.09 | 100.88 | 100.46 | 100.65 |
| 3 | 99.61 | 102.74 | 102.80 | 103.40 | 103.23 | 102.53 |
| 4 | 101.85 | 105.73 | 105.42 | 105.45 | 105.97 | 105.45 |
| 5 | 98.35 | 100.87 | 101.23 | 99.80 | 100.97 | 101.27 |
| 6 | 95.30 | 101.38 | 101.99 | 101.65 | 101.67 | 102.22 |
| Mean (%) | 97.89 | 102.39 | 102.54 | 102.51 | 102.63 | 102.58 |
| RSD (%) | 3.2 | 1.9 | 1.8 | 2.0 | 2.0 | 1.7 |

TABLE 9
Dissolution curve of pimavanserin tartrate capsules
in pH 6.8 phosphate buffer solution

| | Dissolution (%) | | | | | |
|---|---|---|---|---|---|---|
| No. | 5 min | 10 min | 15 min | 20 min | 30 min | 45 min |
| 1 | 93.70 | 101.34 | 102.08 | 101.22 | 102.20 | 102.13 |
| 2 | 96.09 | 102.13 | 102.75 | 103.52 | 102.89 | 101.31 |
| 3 | 93.30 | 101.77 | 102.28 | 101.96 | 101.73 | 101.24 |
| 4 | 94.98 | 103.88 | 104.06 | 104.10 | 104.17 | 104.03 |
| 5 | 93.70 | 102.46 | 102.81 | 103.00 | 103.46 | 102.66 |
| 6 | 95.94 | 103.77 | 104.06 | 105.01 | 104.55 | 103.72 |
| Mean (%) | 94.62 | 102.56 | 103.00 | 103.13 | 103.17 | 102.52 |
| RSD (%) | 1.3 | 1.0 | 0.8 | 1.3 | 1.1 | 1.2 |

TABLE 10
Dissolution curve of Nuplazid ® in 0.1 mol/L
hydrochloric acid solution (literature data)

| | Dissolution (%) | | | | | |
|---|---|---|---|---|---|---|
| No. | 5 min | 10 min | 15 min | 20 min | 30 min | 45 min |
| 1 | 0 | 23 | 67 | 94 | 94 | N/A |
| 2 | 0 | 37 | 82 | 88 | 85 | N/A |
| 3 | 0 | 32 | 86 | 88 | 86 | N/A |
| 4 | 0 | 40 | 84 | 84 | 83 | N/A |
| 5 | 0 | 37 | 85 | 92 | 93 | N/A |
| 6 | 0 | 51 | 86 | 92 | 92 | N/A |
| Mean (%) | 0 | 36.7 | 81.7 | 89.7 | 88.8 | N/A |
| RSD (%) | N/A | 4.0 | 11.1 | 24.4 | 18.9 | N/A |

N/A: Not determined

The experimental results indicated that in these test dissolution media, the preparations prepared by the method of the present invention all realized almost complete dissolution within 5 minutes; whereas, according to the description in the above-mentioned documents, Nuplazid®, a commercially available pimavanserin tartrate capsule, failed to achieve no less than 90% dissolution in 0.1 mol/L hydrochloric acid solution by 30 minutes. This indicated that the dissolution of the solid preparation prepared by the method of the present invention was significantly improved compared with the innovator drug on the market in terms of dissolution.

1. A method for preparing a pimavanserin solid preparation, the method comprising the following steps:
(1) crushing the bulk drug pimavanserin into a particle size of 178 μm or less;
(2) mixing the crushed bulk drug pimavanserin with a pharmaceutically acceptable excipient;
(3) dry granulating the mixed powder; and
(4) pressing the prepared granules into tablets or filling the prepared granules into capsules.

2. The method according to claim 1, wherein the pimavanserin is pimavanserin tartrate.

3. The method according to claim 1, wherein the particle size is expressed as D90, preferably D95, more preferably D98.

4. The method according to claim 1, wherein in step (1), the bulk drug pimavanserin is crushed into a particle size of 170 μm or less, 160 μm or less, or 150 μm or less.

5. The method according to claim 1, wherein the excipient includes a filler (such as microcrystalline cellulose) and a lubricant (such as magnesium stearate).

6. The method according to claim 5, wherein the mass ratio of the bulk drug pimavanserin to the filler to the lubricant is (20-60):(40-80):(0.1-5), preferably (30-50):(50-70):(0.2-2), more preferably about 40:59:1.

7. The method according to claim 1, wherein the mixing is carried out using a mixer (such as a hopper mixer).

8. The method according to claim 7, wherein the rotational speed of the mixer is about 1-100 rpm, and the mixing time is about 1-60 minutes.

9. The method according to claim 1, wherein the dry granulation is carried out using a dry granulator, and the granulation pressure is about 1-100 bar, preferably 20-80 bar, more preferably 40-60 bar, and particularly preferably about 50 bar.

10. The method according to claim 1, wherein after step (3), the following step (3′) is carried out:
(3′) mixing the prepared granules with a second portion of pharmaceutically acceptable excipient, wherein the second portion of pharmaceutically acceptable excipient comprises a second portion of lubricant (such as magnesium stearate).
